(19) United States
(12) Patent Application Publication
(10) Pub. No.: US 2012/0324600 A1
(43) Pub. Date: Dec. 20, 2012

(54) CONSTITUTIVE SYNTHETIC PLANT PROMOTERS AND METHODS OF USE

(75) Inventor: John Daniel Hipskind, Durham, NC (US)

(73) Assignee: SYNGENTA PARTICIPATIONS AG, Basel (CH)

(21) Appl. No.: 13/520,350

(22) PCT Filed: Dec. 13, 2010
(86) PCT No.: PCT/US10/60011

§ 371 Date: Jul. 3, 2012

(30) Foreign Application Priority Data
Jan. 7, 2010 (JP) 2010-002237

(51) Int. Cl. C07H 21/04; C12N 15/82

(52) U.S. Cl. 800/278; 536/24.1; 435/468

(57) ABSTRACT

Control of transgene expression in planta is dependent upon genetic elements that affect both transcription and translation of mRNA transcripts. The disclosed invention describes the combination of DNA elements from four different plant viruses that function as an activator of transcription and enhancer of translation of mRNA transcripts in transgenic plants.

### REFERENCE TO RELATED APPLICATIONS

This is a national stage application under 35 U.S.C. §371 of International Application No. PCT/US 10/60011, filed on Dec. 13, 2010, which is entitled to the benefit of U.S. Provisional Application No. 61/292,239, filed on Jan. 5, 2010, both of which are incorporated herein by reference in their entireties.

### FIELD OF THE INVENTION

The present invention relates to the field of biotechnology, in particular plant biotechnology.

### BACKGROUND

In agricultural biotechnology, plants can be modified according to one's needs. One way to accomplish this is by using modern genetic engineering techniques. For example, by introducing a gene of interest into a plant, the plant can be specifically modified to express a desirable phenotypic trait. For this, plants are transformed most commonly with a heterologous gene comprising a promoter region, a coding region and a termination region. When genetically engineering a heterologous gene for expression in plants, the selection of a promoter is often a critical factor. While it may be desirable to express certain genes constitutively, i.e. throughout the plant at all times and in most tissues and organs, other genes are more desirably expressed only in response to particular stimuli or confined to specific cells or tissues.

It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters”, if the promoters direct RNA synthesis preferentially in certain tissues (RNA synthesis may occur in other tissues at reduced levels). Since patterns of expression of a chimeric gene (or genes) introduced into a plant are controlled using promoters, there is an ongoing interest in the isolation of novel promoters that are capable of controlling the expression of a chimeric gene (or genes) at certain levels in specific tissue types or at specific plant developmental stages.

Certain promoters are able to direct RNA synthesis at relatively similar levels across all tissues of a plant. These are called “constitutive promoters” or “tissue-independent” promoters. Constitutive promoters can be divided into strong, moderate, and weak categories according to their effectiveness to directing RNA synthesis. Since it is necessary in many cases to simultaneously express a chimeric gene (or genes) in different tissues of a plant to get the desired functions of the gene (or genes), constitutive promoters are especially useful in this regard. Though many constitutive promoters have been discovered from plants and plant viruses and characterized, there is still an ongoing interest in the isolation of more novel constitutive promoters, synthetic or native, which are capable of controlling the expression of a chimeric gene (or genes) at different levels and the expression of multiple genes in the same transgenic plant for gene stacking.

Among the most commonly used promoters are the nopaline synthase (NOS) promoter (Ebert et al., Proc. Natl. Acad. Sci. USA 84:5745-5749 (1987)); the octapine synthase (OCS) promoter; caulimovirus promoters such as the cauliflower mosaic virus (CaMV) 19S promoter (Lawton et al., Plant Mol. Biol. 9:315-324 (1987)); the light inducible promoter from the small subunit of rubisco (Pellegrineschi et al., Biochem. Soc. Trans. 23(2):247-250 (1995)); the Adh promoter (Walker et al., Proc. Natl. Acad. Sci. USA 84:6624-66280 (1987)); the sucrose synthase promoter (Yang et al., Proc. Natl. Acad. Sci. USA 87:414-44148 (1990)); the R gene complex promoter (Chandler et al., Plant Cell 1:1175-1183 (1989)); the chlorophyll a/b binding protein gene promoter; and the like.

Homology-dependent gene silencing (HDGS) and homology-dependent male sterility (HDMS) are issues of concern in plant genetic engineering strategies and is thought to be caused by multiple copies of homologous transgene and promoter sequences. Transgene silencing can occur on a transcriptional and post-transcriptional level (Venter, M (2007). Trends Plant Sci. 12(3):1360-1385; Meyer, P and Saedler, H. (1996) Homology dependent gene silencing in plants. Annu. Rev. Plant Physiol. Plant Mol. Biol. 47, 23-48; Kooter, J. M. et al. (1999) Listening to the silent genes: transgene silencing, gene regulation, and pathogen control. Trends Plant Sci. 4, 340-345). Repetitive use of cis-elements with identical core-sequences and homologous intervening regions (within a functional domain) might cause depletion of transcription factors, consequently reducing endogenous gene expression (Bhullar, S. et al. (2003) Strategies for development of functionally equivalent promoters with minimum sequence homology for transgene expression in plant: cis-elements in a novel DNA context versus domain swapping. Plant Physiol. 132, 988-998). Therefore, there is a current need in the industry for synthetic plant promoters capable of expressing heterologous sequences which do not induce HDGS or HDMS. A portion of the synthetic promoters disclosed herein are capable of functioning without the induction of HDGS and HDMS. Additionally, there is a need in the industry to use HDGS or HDMS as a means to select for heterozygous plants in the field. A portion of the synthetic promoters disclosed herein are capable of inducing HDGS and HDMS.

### SUMMARY

One aspect of the present invention is a synthetic plant promoter functional in a plant cell, wherein a 5′ terminus of the synthetic plant promoter is an enhancer from figwort mosaic virus or an enhancer from tobacco mosaic virus and wherein a 3′ terminus of the synthetic plant promoter is an enhancer from the tobacco mosaic virus when the 5′ terminus is the enhancer from figwort mosaic virus or the 3′ terminus is the enhancer from the figwort mosaic virus when the 5′ terminus is the enhancer from the tobacco mosaic virus. In another aspect, the synthetic plant promoter has an optional Kozak sequence which extends beyond the 3′ terminus of the synthetic plant promoter. In another aspect of the synthetic plant promoter, the enhancer from a figwort mosaic virus comprises SEQ ID NO: 1 and the enhancer from a tobacco mosaic virus comprises SEQ ID NO: 2. In yet another aspect of the present invention, the synthetic plant promoter comprises SEQ ID NO: 3. In still yet another aspect, the synthetic plant promoter comprises SEQ ID NO: 4. In further still another aspect of the present invention, the synthetic plant promoter comprises SEQ ID NO: 5. In another aspect, the synthetic plant promoter comprises SEQ ID NO: 6. In still yet another aspect, the synthetic plant promoter comprises SEQ ID NO: 7. In further yet another aspect, the synthetic plant promoter comprises SEQ ID NO: 8. In still yet another aspect, the synthetic plant promoter comprises SEQ ID NO: 9.

Another aspect of the present invention is a method of constructing a synthetic plant promoter functional in a plant comprising the steps of (a) obtaining an enhancer from a figwort mosaic virus and an enhancer from a tobacco mosaic virus and optionally one or more nucleotide sequences selected from the group consisting of enhancers, promoters, exons, introns, and other regulatory sequences; (b) operably linking the enhancer from the figwort mosaic virus, the one or more optional nucleotide sequences, and the enhancer from the tobacco mosaic virus thus creating the synthetic plant promoter functional in a plant, wherein a 5′ terminus of the synthetic plant promoter is the enhancer from figwort mosaic virus or the enhancer from tobacco mosaic virus and wherein a 3′ terminus of the synthetic plant promoter is the enhancer from a tobacco mosaic virus when the said 5′ terminus is the enhancer from the figwort mosaic virus or the 3′ terminus of the synthetic plant promoter is the enhancer from the figwort mosaic virus when the said 5′ terminus is the enhancer from the tobacco mosaic virus, and wherein the one or more optional nucleotide sequences are positioned between the enhancers. Yet another aspect of the present invention, the enhancer from the figwort mosaic virus comprises SEQ ID NO: 1 and the enhancer from the tobacco mosaic virus comprises SEQ ID NO: 2. In still yet another aspect, the product of step (b) comprises SEQ ID NO: 3. In another aspect of the present invention, the product of step (b) comprises SEQ ID NO: 4. In yet another aspect, the product of step (b) comprises SEQ ID NO: 5. In still yet another aspect, the product of step (b) comprises SEQ ID NO: 6. In yet another aspect, the product of step (b) comprises SEQ ID NO: 7. In further yet another aspect, the product of step (b) comprises SEQ ID NO: 8. In still yet another aspect, the product of step (b) comprises SEQ ID NO: 9.

Yet another aspect of the present invention is a method of expressing a heterologous gene in a plant, plant cell, or plant tissue, comprising (a) constructing an expression cassette according to the method above, wherein the expression cassette is functional in a plant, plant cell, or plant tissue; and (b) creating a plant, plant cell, or plant tissue or a portion thereof comprising the expression cassette, wherein the heterologous gene is expressed. In another aspect, the heterologous gene comprises a nucleotide sequence encoding an herbicide resistance trait. In yet another aspect, the nucleotide sequence encoding an herbicide resistance trait comprises a nucleotide sequence encoding HPPD resistance. In still yet another aspect, the synthetic plant promoter is manipulated to optimize expression. In yet another aspect, the synthetic plant promoter is manipulated to reduce expression. In another aspect, the synthetic plant promoter is manipulated to increase expression. In yet another aspect, the plant, plant cell, or plant tissue or a portion thereof is a monocot. In still yet another aspect, the plant, plant cell, or plant tissue or a portion thereof is maize. In further yet another aspect, the plant, plant cell, or plant tissue or a portion thereof is a dicot. In still yet another aspect, the plant, plant cell, or plant tissue or a portion thereof is soybean.

Another aspect of the present invention is a method of selecting for male sterile plants comprising: (a) constructing an expression cassette comprising a synthetic plant promoter operably linked to a heterologous gene, wherein a 5′ terminus of the synthetic plant promoter comprises SEQ ID NO: 1 or SEQ ID NO: 2 and wherein a 3′ terminus of the synthetic plant promoter comprises SEQ ID NO: 2 when the 5′ terminus is SEQ ID NO: 1 or the 3′ terminus of the synthetic plant promoter is SEQ ID NO: 1 when the 5′ terminus is SEQ ID NO: 2, and wherein the synthetic plant promoter is functional in a plant cell; (b) creating a plant, plant cell, or plant tissue or a portion thereof comprising the expression cassette, wherein the heterologous gene is overexpressed and wherein such overexpression induces male sterility; and (c) selecting for the male sterile plants. In another aspect, the synthetic plant promoter is selected from the group consisting of: SEQ ID NOs: 4 and 6. In yet another aspect, the heterologous gene comprises a nucleotide sequence encoding an herbicide resistance trait. In still yet another aspect, the nucleotide sequence encoding an herbicide resistance trait comprises a nucleotide sequence encoding HPPD resistance.

Yet another aspect of the present invention is a method of selecting for heterozygous plants comprising: (a) constructing an expression cassette comprising a synthetic plant promoter operably linked to a heterologous gene, wherein a 5′ terminus of the synthetic plant promoter comprises SEQ ID NO: 1 or SEQ ID NO: 2 and wherein a 3′ terminus of the synthetic plant promoter comprises SEQ ID NO: 2 when the 5′ terminus is SEQ ID NO: 1 or the 3′ terminus of the synthetic plant promoter is SEQ ID NO: 1 when the 5′ terminus is SEQ ID NO: 2, and wherein the synthetic plant promoter is functional in a plant cell; (b) creating a plant, plant cell, or plant tissue or a portion thereof comprising the expression cassette, wherein the heterologous gene is overexpressed in homozygous plants and wherein such overexpression induces gene silencing; and (c) selecting for the heterozygous plants. In another aspect, the synthetic plant promoter is selected from the group consisting of: SEQ ID NOs: 4 and 6. In yet another aspect, the heterologous gene comprises a nucleotide sequence encoding an herbicide resistance trait. In further yet another aspect, the nucleotide sequence encoding an herbicide resistance trait comprises a nucleotide sequence encoding HPPD resistance.

These and other features, aspects, and advantages of the present invention will become better understood with reference to the following description and appended claims.

### BRIEF DESCRIPTION OF THE SEQUENCES IN THE SEQUENCE LISTING

SEQ ID NO: 1 is the nucleotide sequence of the figwort mosaic virus enhancer eFMV-03.

SEQ ID NO: 2 is the nucleotide sequence of the tobacco mosaic virus enhancer eTMV-02.

SEQ ID NO: 3 is the nucleotide sequence of a synthetic plant promoter.

SEQ ID NO: 4 is the nucleotide sequence of a synthetic plant promoter.

SEQ ID NO: 5 is the nucleotide sequence of a synthetic plant promoter.

SEQ ID NO: 6 is the nucleotide sequence of a synthetic plant promoter.

SEQ ID NO: 7 is the nucleotide sequence of a synthetic plant promoter.

SEQ ID NO: 8 is the nucleotide sequence of a synthetic plant promoter.

SEQ ID NO: 9 is the nucleotide sequence of a synthetic plant promoter.

SEQ ID NO: 10 is the nucleotide sequence of a wildtype cestrum virus promoter.

### DEFINITIONS

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

The term “nucleic acid” refers to a polynucleotide of high molecular weight which can be single-stranded or double-stranded, composed of monomers (nucleotides) containing a sugar, phosphate and a base which is either a purine or pyrimidine. A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. A “genome” is the entire body of genetic material contained in each cell of an organism. The term “nucleotide sequence” refers to a polymer of DNA or RNA which can be single- or double-stranded, optionally containing synthetic, non-natural or altered nucleotide bases capable of incorporation into DNA or RNA polymers. Unless otherwise indicated, a particular nucleic acid sequence of this invention also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer, et al., Nucleic Acid Res. 19:5081 (1991); Ohtsuka, et al., J. Biol. Chem. 260:2605-2608 (1985); and Rossolini, et al., Mol. Cell. Probes 8:91-98 (1994)). The term nucleic acid is used interchangeably with gene, cDNA, and mRNA encoded by a gene.

“Operably-linked” refers to the association of nucleic acid sequences on a single nucleic acid fragment so that the function of one is affected by the other. For example, a promoter is operably-linked with a coding sequence or functional RNA when it is capable of affecting the expression of that coding sequence or functional RNA (i.e., that the coding sequence or functional RNA is under the transcriptional control of the promoter). Coding sequences in sense or antisense orientation can be operably-linked to regulatory sequences.

“Promoter” refers to a nucleotide sequence which controls the expression of a coding sequence by providing the recognition for RNA polymerase and other factors required for proper transcription. “Promoter regulatory sequences” consist of proximal and more distal upstream elements. Promoter regulatory sequences influence the transcription, RNA processing or stability, or translation of the associated coding sequence. Regulatory sequences include enhancers, untranslated leader sequences, introns, and polyadenylation signal sequences. They include natural and synthetic sequences as well as sequences that may be a combination of synthetic and natural sequences. The meaning of the term “promoter” includes “promoter regulatory sequences.”

An “enhancer” is a nucleotide sequence that can stimulate promoter activity and can be an innate element of the promoter or a heterologous element inserted to enhance the level or tissue specificity of a promoter. The primary sequence can be present on either strand of a double-stranded DNA molecule, and is capable of functioning even when placed either upstream or downstream from the promoter. A “transcriptional enhancer” functions in that it increases the amount of messenger RNA (mRNA) transcript which is translated from the DNA molecule. A “translational enhancer” functions in that it increases the amount of protein translated from the mRNA molecule.

“Gene” refers to a nucleic acid fragment that expresses mRNA, functional RNA, or specific protein, including regulatory sequences. The term “Native gene” refers to a gene as found in nature. The term “chimeric gene” refers to any gene that contains 1) DNA sequences, including regulatory and coding sequences, that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature. A “transgene” refers to a gene that has been introduced into the genome by transformation and is stably maintained. Transgenes may include, for example, genes that are either heterologous or homologous to the genes of a particular plant to be transformed. Additionally, transgenes may comprise native genes inserted into an organism. Transgenes may be chimeric genes. The term “endogenous gene” refers to a native gene in its natural location in the genome of an organism. A “foreign” gene refers to a gene not normally found in the host organism but one that is introduced into the organism by gene transfer.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components.

“Intron” refers to an intervening section of DNA which occurs almost exclusively within a eukaryotic gene, but which is not translated to amino acid sequences in the gene product. The introns are removed from the pre-mature mRNA through a process called splicing, which leaves the exons untouched, to form an mRNA. For purposes of the present invention, the definition of the term “intron” includes modifications to the nucleotide sequence of an intron derived from a target gene, provided the modified intron does not significantly reduce the activity of its associated 5′ regulatory sequence.

“Exon” refers to a section of DNA which carries the coding sequence for a protein or part of it. Exons are separated by intervening, non-coding sequences (introns). For purposes of the present invention, the definition of the term “exon” includes modifications to the nucleotide sequence of an exon derived from a target gene, provided the modified exon does not significantly reduce the activity of its associated 5′ regulatory sequence.

Expression or overexpression of a gene involves transcription of the gene and translation of the mRNA into a precursor or mature protein. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression or transcript accumulation of identical or substantially similar foreign or endogenous genes. The mechanism of co-suppression may be at the DNA level (such as DNA methylation), at the transcriptional level, or at post-transcriptional level.

The term “constitutive promoter” refers to a promoter active in all or most tissues of a plant at all or more developing stages. As with other promoters classified as constitutive, some variation in absolute levels of expression can exist among different tissues or stages.

The term “constitutive promoter” or “tissue-independent” are used interchangeably herewithin.

The term “isolated” when used in relation to a nucleic acid refers to a nucleic acid sequence that is identified and separated from at least one contaminant nucleic acid with which it is ordinarily associated in its natural source. An isolated nucleic acid is present in a form or setting that is different from that in which it is found in nature. In contrast, a non-isolated nucleic acids such as DNA and RNA found in the state they exist in nature. An isolated nucleic acid may be in a transgenic plant and still be considered “isolated”.

The terms “polynucleotide”, “polynucleotide sequence”, “nucleic acid sequence”, and “nucleic acid fragment”/“isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

A “heterologous nucleic acid fragment” refers to a sequence that is not naturally occurring with the synthetic plant promoter sequence of the invention. While this nucleotide sequence is heterologous to the promoter sequence, it may be homologous, or native, or heterologous, or foreign, to the plant host.

The term “substantially similar” as used herein refers to nucleic acid fragments wherein changes in one or more nucleotide bases do not affect the ability of the nucleic acid fragment to mediate gene expression or produce a certain phenotype. This term also refers to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences.

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., Plant Cell 1:671-680 (1989).

“Transformation” refers to the transfer of a nucleic acid fragment into the genome of a host organism, resulting in genetically stable inheritance. Host organisms containing the transformed nucleic acid fragments are referred to as “transgenic” organisms.

“Transient expression” refers to the temporary expression of often reporter genes such as β-glucuronidase (GUS), fluorescent protein genes GFP, ZS-YELLOW1 N1, AM-CYAN1, DS-RED in selected certain cell types of the host organism in which the transgenic gene is introduced temporally by a transformation method.

Standard recombinant DNA and molecular cloning techniques used herein are well known in the art and are described more fully in Sambrook, J. et al., In Molecular Cloning: A Laboratory Manual; 2^{nd} ed.; Cold Spring Harbor Laboratory Press: Cold Spring Harbor, N.Y., 1989 (hereinafter “Sambrook et al., 1989”) or Ausubel, F. M., Brent, R., Kingston, R. E., Moore, D. D., Seidman, J. G., Smith, J. A. and Struhl, K., Eds.; In Current Protocols in Molecular Biology; John Wiley and Sons: New York, 1990 (hereinafter “Ausubel et al., 1990”).

“PCR” or “Polymerase Chain Reaction” is a technique for the synthesis of large quantities of specific DNA segments, consisting of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps comprises a cycle.

### DETAILED DESCRIPTION

The synthetic plant promoter nucleotide sequences and methods disclosed herein are useful in regulating expression of any heterologous nucleic acid sequences in a host plant in order to alter the phenotype of a plant.

Various changes in phenotype are of interest including, but not limited to, modifying the fatty acid composition in a plant, altering the amino acid content of a plant, altering a plant's pathogen defense system, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic characteristics and traits such as yield and heterosis increase, the choice of genes for transformation will change accordingly. Categories of transgenes, also known as heterologous genes, for example, include, but are not limited to, genes encoding important agronomic traits, insect resistance, disease resistance, herbicide resistance, sterility, grain or seed characteristics, and commercial products. Genes of interest include, generally, those involved in oil, starch, carbohydrate, or nutrient metabolism as well as those affecting seed size, plant development, plant growth regulation, and yield improvement. Plant development and growth regulation also refer to the development and growth regulation of various parts of a plant, such as the flower, seed, root, leaf, and shoot.

Other commercially desirable traits are genes and proteins conferring cold, heat, salt, and drought resistance.

Disease and/or insect resistance genes may encode resistance to pests that have great yield drag such as for example, anthracnose, soybean mosaic virus, soybean cyst nematode, root-knot nematode, brown leaf spot, Downy mildew, purple seed stain, seed decay, and seedling diseases commonly caused by the fungi Pythium sp., Phytophthora sp., Rhizoctonia sp., Diaporthe sp. Bacterial blight caused by the bacterium Pseudomonas syringae pv. Glycinea. Genes conferring insect resistance include, for example, Bacillus thuringiensis toxic protein genes (U.S. Pat. Nos. 5,366,892; 5,747,450; 5,737,514; 5,723,756; 5,593,881; and Geiser et al (1986) Gene 48:109); lectins (Van Damme et al. (1994) Plant Mol. Biol. 24:825); vegetative insecticidal proteins (VIP3C, U.S. Pat. No. 7,378,493); and the like.

Herbicide resistance traits may include genes coding for resistance to herbicides that act to inhibit the action of acetolactate synthase (ALS), in particular the sulfonylurea-type herbicides (e.g., the acetolactate synthase ALS gene containing mutations leading to such resistance, in particular the S4 and/or HRA mutations). The ALS-gene mutants encode resistance to the herbicide chlorosulfuron. Glyphosate acetyl transferase (GAT) is an N-acetyltransferase from Bacillus licheniformis that was optimized by gene shuffling for acetylation of the broad spectrum herbicide, glyphosate, forming the basis of a novel mechanism of glyphosate tolerance in transgenic plants (Castle et al. (2004) Science 304, 1151-1154). Other herbicide resistance traits, including, but not limited to, EPSPS (U.S. Pat. No. 6,248,076), Bar (U.S. Pat. No. 6,025,545), and HPPD (U.S. Pat. No. 7,312,379), would be obvious to use to one skilled in the art.

The present invention includes the transformation of a recipient cell with at least one advantageous transgene. Two or more transgenes can be supplied in a single transformation event using either distinct transgene-encoding vectors, or a single vector incorporating two or more gene coding sequences. Any two or more transgenes of any description, such as those conferring herbicide, insect, disease (viral, bacterial, fungal, and nematode) or drought resistance, oil quantity and quality, or those increasing yield or nutritional quality may be employed as desired.

The synthetic plant promoter sequence of the present invention can be modified to provide a range of constitutive expression levels of the heterologous nucleotide sequence. Thus, less than the entire synthetic plant promoter regions may be utilized and the ability to drive expression of the coding sequence retained. However, it is recognized that expression levels of the mRNA may be decreased with deletions of portions of the synthetic plant promoter sequences. Therefore, fragments of SEQ ID NO: 3 which are 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 3 may still function as exemplified by this description.

Embraced by the present invention are also functional equivalents of the synthetic plant promoters of the present invention, i.e. nucleotide sequences that hybridize under stringent conditions to any one of SEQ ID NO:3, SEQ ID NO:4, SEQ ID NO: 5, SEQ ID NO:6, SEQ ID NO: 7, SEQ ID NO: 8, or SEQ ID NO: 9. A stringent hybridization is performed at a temperature of 65° C., preferably 60° C. and most preferably 55° C. in double strength (2×) citrate buffered saline (SSC) containing 0.1% SDS followed by rinsing of the support at the same temperature but with a buffer having a reduced SSC concentration. Such reduced concentration buffers are typically one tenth strength SSC (0.1×SSC) containing 0.1% SDS, preferably 0.2×SSC containing 0.1% SSC and most preferably half strength SSC (0.5×SSC) containing 0.1% SDS.

### EXAMPLES

### Example 1

### Combining Viral Enhancer Elements and Plant Components to Create Synthetic Plant Promoters

To express a synthetic oat (Avena sativa) 4-hydroxyphenylpyruvate dioxygenase (cAvHPPD) in stably transformed soybean, viral transcriptional and translational enhancers, and a minimal promoter were created by PCR or direct DNA synthesis and combined by standard DNA restriction digestion and ligation reactions. A synthetic plant promoter comprising defined components eFMV (SEQ ID NO: 1), eTMV (SEQ ID NO: 2), the Cauliflower Mosaic Virus 35S enhancer region (e35S) and a minimal promoter (pr35SCMP: Cestrum Yellow Leaf Curl virus TATA-box motif; no CAAT 35S-proximal promoter sequence) were combined to create SEQ ID NO: 3. Subsequently, SEQ ID NO: 3 was modified by digestion with a DNA restriction enzyme XhoI to remove defined components e35S, pr35SCMP (including the TATA-box motif) followed by a standard ligation reaction to create SEQ ID NO: 4. The SEQ ID NO: 3 was again modified by the ligation of the first intron (iUBQ3) derived from the Arabidopsis ubiquitin promoter as a Bgl II (5-prime end) and BamHI (3-prime end) DNA fragment to the BamHI site to create SEQ ID NO: 5. Finally, SEQ ID NO: 3 was modified by the ligation of a 1092 base pair DNA fragment of an Arabidopsis constitutive promoter (prAC26) as a Bgl II (5-prime end) and BamHI (3-prime end) to the BamHI site to create SEQ ID NO: 6. The completed gene cassettes harboring individual synthetic plant promoters comprising SEQ ID: 3, SEQ ID: 4, SEQ ID: 5 or SEQ ID: 6, the cAvHPPD coding region and NOS terminator (tNOS) were subsequently ligated to binary vectors containing the appropriate selectable markers for soybean transformation experiments. Table 1 indicates the arrangement of subelements in the above described synthetic plant promoters. One skilled in the art would readily recognize other subelements which would be suitable to use.

TABLE 1
5′ to 3′ arrangement of subelements in synthetic plant promoters for soy.

| SEQ ID | | | | | | | length |
|---|---|---|---|---|---|---|---|
| NO: | Subelements | | | | | | (bp) |
| 3 | eFMV | e35S | — | — | pr35SCMP | eTMV | 625 |
| 4 | eFMV | — | — | — | — | eTMV | 267 |
| 5 | eFMV | e35S | — | iUBQ3 | pr35SCMP | eTMV | 1017 |
| 6 | eFMV | e35S | prAC26 | — | pr35SCMP | eTMV | 1716 |
| 7 | eFMV | e35S | prAC26 | iUBQ3 | pr35SCMP | eTMV | 2108 |

The plasmids containing the synthetic plant promoter expression cassettes were transformed into soybean using Agrobacterium tumefaciens. T0 events were cultivated and selected for cAvHPPD expression by application of mesotrione spray. Leaf samples of surviving T0 plants were tested for zygosity by TaqMan® assay. Expression of cAvHPPD of surviving T0 plants was quantified by ELISA (Engvall E, Perlman P (1971). “Enzyme-linked immunosorbent assay (ELISA). Quantitative assay of immunoglobulin G”. Immunochemistry 8(9):871-874).

### Example 2

### Transgenic Soybean Event Characterization

The first generation transgenic soybean events (T1) harboring SEQ ID NO: 3, SEQ ID NO: 4, SEQ ID NO: 5, SEQ ID NO: 6, or SEQ ID NO: 7 were characterized for segregation analysis, oat HPPD protein expression and tolerance to mesotrione herbicide spray. The green leaf tissues from the first trifoliate of five independent events were sampled to determine the segregation ratios (homozygous, heterozygous or null) of the individual seedlings as determined by zygosity Taqman® assays and oat HPPD protein expression by ELISA. At the V2 stage, the seedlings were sprayed with the HPPD inhibiting herbicide mesotrione and tolerance rating determined approximately 10 days post-application. The results from analysis of transgenic soybean events harboring SEQ ID NO: 3 showed significant initial damage to the homozygous (HOM) compared to the heterozygous (HET) siblings. These data are consistent with the ELISA data showing relatively low level of oat HPPD protein expression in the HOM seedlings (<20 ng/mg total protein) compared to HET siblings for each independent event (Table 2). Collectively, these results are indicative of transgene silencing whereby overexpression of a transgene in HOM siblings activates a micro-RNA mediated methylation which results in very low expression of the transgene (Martienssen R A, Colot V (2001). DNA Methylation and Epigenetic Inheritance in Plants and Filamentous Fungi. Science 293(5532):1070-1074). The analyses of T1 generation soybean events harboring SEQ ID NO: 4 or SEQ ID NO: 5 showed more consistent expression of oat HPPD protein as would be expected in HOM siblings (Table 3, Table 4, respectively). These data reveal that SEQ ID NO: 4 and SEQ ID NO: 5 modulate oat HPPD expression to such an extent as to relieve transgene silencing resulting and improved tolerance to the HPPD inhibitor herbicide mesotrione. However, the modification as exemplified in SEQ ID NOs: 6 and 7 did not relieve transgene silencing in HOM plants (Tables 5 and 6).

TABLE 2
SEQ ID NO: 3

| | | ELISA (ng/mg |
|---|---|---|
| Plant Event ID | Zygosity | total protein) |
| SYHT2007020113A010A~9 | HET | 1946 |
| SYHT2007020113A010A~11 | HOM | <20 |
| SYHT2007020085B011A~3 | HET | 1060 |
| SYHT2007020085B011A~2 | HOM | <20 |
| SYHT2007020085A021A~5 | HET | 1255 |
| SYHT2007020085A021A~7 | HOM | <20 |
| SYHT2007020113A014A~5 | HET | 1105 |
| SYHT2007020113A014A~4 | HOM | 23 |
| SYHT2007019943A004A~22 | HET | 1956 |
| SYHT2007019943A004A~25 | HOM | 298 |

TABLE 3
SEQ ID NO: 4

| | | ELISA (ng/mg |
|---|---|---|
| Plant Event ID | Zygosity | total protein) |
| SYHT080447A002A~3 | HET | 97.38 |
| SYHT080447A002A~10 | HOM | 161.78 |
| SYHT080508A002A~7 | HET | 124.95 |
| SYHT080508A002A~10 | HOM | 356 |
| SYHT080523B003A~4 | HET | 135.96 |
| SYHT080523B003A~3 | HOM | 255.78 |
| SYHT080514A003A~11 | HET | 129.23 |
| SYHT080514A003A~7 | HOM | 214.5 |
| SYHT080508A004A~7 | HET | 198.23 |
| SYHT080508A004A~6 | HOM | 312.76 |

TABLE 4
SEQ ID NO: 5

| | | ELISA (ng/mg |
|---|---|---|
| Plant Event ID | Zygosity | total protein) |
| SYHT080552B001A~3 | HET | 521.62 |
| SYHT080552B001A~2 | HOM | 849.12 |
| SYHT080567A005A~10 | HET | 269.2 |
| SYHT080567A005A~11 | HOM | 515.89 |
| SYHT080551A003A~9 | HET | 280.1 |
| SYHT080551A003A~10 | HOM | 658.14 |
| SYHT080551A007A~9 | HET | 220.59 |
| SYHT080551A007A~8 | HOM | 610.08 |
| SYHT080553A002A~6 | HET | 231.28 |
| SYHT080553A002A~5 | HOM | 412.1 |

TABLE 5
SEQ ID NO: 6

| | | ELISA (ng/mg |
|---|---|---|
| Plant Event ID | Zygosity | total protein) |
| SYHT080609A001A~10 | HET | 1345 |
| SYHT080609A001A~1 | HOM | 231.95 |
| SYHT080609B001A~1 | HET | 1070 |
| SYHT080609B001A~10 | HOM | 206.2 |
| SYHT080601A009A~11 | HET | 1635 |
| SYHT080601A009A~12 | HOM | 90.4 |
| SYHT080570A005A~6 | HET | 2325 |
| SYHT080570A005A~5 | HOM | 713.2 |
| SYHT080601A003A~10 | HET | 1862.4 |
| SYHT080601A003A~8 | HOM | 88.62 |

TABLE 6
SEQ ID NO: 7

| | | ELISA (ng/mg |
|---|---|---|
| Plant Event ID | Zygosity | total protein) |
| SYHT080721B001A~3 | Het | 961.967 |
| SYHT080721B001A~8 | Hom | 97.726 |
| SYHT080723A001A~4 | Het | 650.125 |
| SYHT080723A001A~7 | Hom | 125.373 |
| SYHT080723B004A~9 | Het | 1046.682 |
| SYHT080723B004A~3 | Hom | 137.826 |
| SYHT080749B004A~3 | Het | 704.445 |
| SYHT080749B004A~10 | Hom | 67.075 |
| SYHT080723B005A~6 | Het | 613.129 |
| SYHT080723B005A~10 | Hom | 401.003 |

### Example 3

### Transgenic Corn Event Characterization

A similar strategy for building synthetic plant promoters for use in maize was implemented. In addition to the promoters illustrated in Table 7, below, SEQ ID NO: 3 was also successfully used to promote the expression of a heterologous sequence in maize.

TABLE 7
5′ to 3′ arrangement of subelements in synthetic plant promoters for maize.

| SEQ ID | | | | | | | | length |
|---|---|---|---|---|---|---|---|---|
| NO: | Subelements¹ | | | | | | | (bp) |
| 8 | eFMV | e35S | — | — | — | prTaHisH3 | eTMV | 834 |
| 9 | eFMV | e35S | eNOS | prCMP | xZmH3Cis | — | eTMV | 1294 |

¹For SEQ ID NOs: 8 and 9, a Kozak sequence is located between the 3′ terminus of the eTMV subelement and the start codon of the heterologous gene.

SEQ ID NO: 8 was synthesized by Gene Art as a SanDI/BamHI fragment then ligated directly into a cloning vector harboring the EPSPS gene (cZmEPSPSct-01) to confer glyphosate tolerance (Terada, et al., (1995) A type I element composed of the hexamer (ACGTCA) and octamer (CGCGGATC) motifs plays a role(s) in meristematic expression of a wheat histone H3 gene in transgenic rice plants. Plant Molecular Biology 27: 17-26). SEQ ID NO: 9 was created by ligation of the xZmH3Cis DNA elements to the prCMP promoter as an NheI fragment such that these elements are 5′ to the TATA-BOX (Brignon, et al., (1993) Nuclease sensitivity and functional analysis of a maize histone H3 gene promoter. Plant Molecular Biology 22: 1007-1015).

Data indicate that SEQ ID NOs: 8 and 9 were as efficient in promoting the expression of an operably linked heterologous sequence as an unmodified cestrum virus promoter (SEQ ID NO: 10). See Table 9 for glyphosate phytotoxicity, in terms of percent injury. Plants were sprayed with an appropriate amount of glyphosate (i.e. 4× Touchdown®) at the V4 stage and the V8 stage. Percent injury was measured at 7 and 14 days after the V4 stage glyphosate spray, as well as 7 and 14 days after V8 stage glyphosate spray.

TABLE 9
Glyphosate Phytotoxicity (Percent Injury)

| | | | 14 d | | 14 d |
|---|---|---|---|---|---|
| | | 7 d after | after | 7 d after | after |
| | SEQ ID | 4X V4 | 4X V4 | 4X V8 | 4X V8 |
| Plant Name | NO: | spray | spray | spray | spray |
| NEG(N)/MZHG032(S) | 10 | 22.50 | 25.00 | 22.50 | 25.00 |
| NEG(N)/MZHG033(S) | 10 | 17.50 | 15.00 | 20.00 | 20.00 |
| NEG(N)/MZHG036(S) | 10 | 20.00 | 25.00 | 20.00 | 17.50 |
| NEG(N)/MZHG037(S) | 10 | 17.50 | 22.50 | 25.00 | 25.00 |
| NEG(N)/MZHG038(S) | 10 | 20.00 | 15.00 | 17.50 | 20.00 |
| NEG(N)/MZHG03C(S) | 10 | 20.00 | 22.50 | 22.50 | 20.00 |
| NEG(N)/MZHG03D(S) | 10 | 12.50 | 30.00 | 15.00 | 17.50 |
| NEG(N)/MZHG03E(S) | 10 | 10.00 | 22.50 | 25.00 | 25.00 |
| NEG(N)/MZHG03F(S) | 10 | 35.00 | 25.00 | 32.50 | 30.00 |
| NEG(N)/MZHG03R(S) | 10 | 15.00 | 20.00 | 20.00 | 20.00 |
| NEG(N)/MZHG03S(S) | 10 | 60.00 | 70.00 | 65.00 | 65.00 |
| NEG(N)/MZHG03X(S) | 10 | 5.00 | 12.50 | 12.50 | 10.00 |
| NEG(N)/MZHG03Y(S) | 10 | 40.00 | 50.00 | 40.00 | 45.00 |
| NEG(N)/MZHG03Z(S) | 10 | 30.00 | 40.00 | 30.00 | 35.00 |
| NEG(N)/MZHG040(S) | 10 | 40.00 | 40.00 | 35.00 | 42.50 |
| NEG(N)/MZHG041(S) | 10 | 20.00 | 22.50 | 22.50 | 22.50 |
| NEG(N)/MZHG04A(S) | 10 | 17.50 | 17.50 | 15.00 | 17.50 |
| NEG(N)/MZHG04D(S) | 10 | 10.00 | 20.00 | 17.50 | 15.00 |
| NEG(N)/MZHG022(S) | 8 | 15.00 | 15.00 | 15.00 | 17.50 |
| NEG(N)/MZHG02D(S) | 8 | 17.50 | 25.00 | 20.00 | 20.00 |
| NEG(N)/MZHG02P(S) | 8 | 25.00 | 22.50 | 25.00 | 30.00 |
| NEG(N)/MZHG02S(S) | 8 | 12.50 | 30.00 | 30.00 | 32.50 |
| NEG(N)/MZHG04E(S) | 8 | 25.00 | 15.00 | 22.50 | 25.00 |
| NEG(N)/MZHG04F(S) | 8 | 25.00 | 20.00 | 22.50 | 22.50 |
| NEG(N)/MZHG04G(S) | 8 | 50.00 | 70.00 | 60.00 | 60.00 |
| NEG(N)/MZHG04M(S) | 8 | 12.50 | 17.50 | 17.50 | 22.50 |
| NEG(N)/MZHG04P(S) | 8 | 20.00 | 20.00 | 20.00 | 22.50 |
| NEG(N)/MZHG04T(S) | 8 | 20.00 | 20.00 | 20.00 | 25.00 |
| NEG(N)/MZHG04V(S) | 8 | 27.50 | 37.50 | 30.00 | 35.00 |
| NEG(N)/MZHG04X(S) | 8 | 27.50 | 40.00 | 30.00 | 32.50 |
| NEG(N)/MZHG04Y(S) | 8 | 80.00 | 90.00 | 90.00 | 90.00 |
| NEG(N)/MZHG051(S) | 8 | 15.00 | 25.00 | 20.00 | 20.00 |
| NEG(N)/MZHG053(S) | 8 | 40.00 | 50.00 | 42.50 | 45.00 |
| One | | | | | |
| NEG(N)/MZHG053(S) | 8 | 40.00 | 50.00 | 42.50 | 42.50 |
| Two | | | | | |
| NEG(N)/MZHG057(S) | 8 | 40.00 | 40.00 | 35.00 | 42.50 |
| NEG(N)/MZHG05E(S) | 8 | 15.00 | 15.00 | 17.50 | 17.50 |
| NEG(N)/MZHG05J(S) | 8 | 15.00 | 17.50 | 22.50 | 20.00 |
| NEG(N)/MZHG05L(S) | 8 | 10.00 | 10.00 | 12.50 | 15.00 |
| NEG(N)/MZHG05M(S) | 8 | 30.00 | 20.00 | 32.50 | 32.50 |
| NEG(N)/MZHG05N(S) | 8 | 10.00 | 5.00 | 10.00 | 10.00 |
| NEG(N)/MZHG05O(S) | 8 | 20.00 | 15.00 | 20.00 | 20.00 |
| NEG(N)/MZHG05P(S) | 8 | 22.50 | 17.50 | 22.50 | 22.50 |
| NEG(N)/MZHG05Q(S) | 8 | 15.00 | 25.00 | 20.00 | 20.00 |
| NEG(N)/MZHG05S(S) | 8 | 17.50 | 12.50 | 15.00 | 17.50 |
| NEG(N)/MZHG05V(S) | 8 | 80.00 | 90.00 | 90.00 | 90.00 |
| NEG(N)/MZHG05W(S) | 8 | 17.50 | 20.00 | 17.50 | 17.50 |
| NEG(N)/MZHG05X(S) | 8 | 12.50 | 15.00 | 17.50 | 17.50 |
| NEG(N)/MZHG061(S) | 8 | 20.00 | 15.00 | 17.50 | 17.50 |
| NEG(N)/MZHG062(S) | 8 | 25.00 | 17.50 | 22.50 | 22.50 |
| NEG(N)/MZHG063(S) | 8 | 17.50 | 10.00 | 17.50 | 17.50 |
| NEG(N)/MZHG064(S) | 8 | 10.00 | 17.50 | 17.50 | 17.50 |
| NEG(N)/MZHG065(S) | 8 | 17.50 | 17.50 | 17.50 | 22.50 |
| NEG(N)/MZHG066(S) | 8 | 20.00 | 20.00 | 20.00 | 20.00 |
| NEG(N)/MZHG06C(S) | 8 | 27.50 | 30.00 | 30.00 | 30.00 |
| NEG(N)/MZHG06H(S) | 9 | 12.50 | 20.00 | 15.00 | 17.50 |
| NEG(N)/MZHG06I(S) | 9 | 17.50 | 20.00 | 22.50 | 25.00 |
| NEG(N)/MZHG06J(S) | 9 | 15.00 | 12.50 | 20.00 | 22.50 |
| NEG(N)/MZHG06M(S) | 9 | 30.00 | 35.00 | 35.00 | 37.50 |
| NEG(N)/MZHG06N(S) | 9 | 27.50 | 25.00 | 27.50 | 30.00 |
| NEG(N)/MZHG06O(S) | 9 | 20.00 | 17.50 | 20.00 | 22.50 |
| NEG(N)/MZHG06P(S) | 9 | 15.00 | 17.50 | 17.50 | 17.50 |
| NEG(N)/MZHG06Q(S) | 9 | 15.00 | 20.00 | 22.50 | 20.00 |
| NEG(N)/MZHG06S(S) | 9 | 70.00 | 80.00 | 80.00 | 80.00 |
| NEG(N)/MZHG06U(S) | 9 | 25.00 | 20.00 | 22.50 | 22.50 |
| NEG(N)/MZHG06V(S) | 9 | 35.00 | 35.00 | 30.00 | 35.00 |
| NEG(N)/MZHG06X(S) | 9 | 10.00 | 15.00 | 10.00 | 15.00 |
| NEG(N)/MZHG06Z(S) | 9 | 5.00 | 7.50 | 10.00 | 12.50 |
| NEG(N)/MZHG072(S) | 9 | 0.00 | 7.50 | 0.00 | 5.00 |
| NEG(N)/MZHG073(S) | 9 | 20.00 | 20.00 | 20.00 | 20.00 |
| NEG(N)/MZHG074(S) | 9 | 15.00 | 20.00 | 20.00 | 20.00 |
| NEG(N)/MZHG078(S) | 9 | 20.00 | 35.00 | 27.50 | 32.50 |
| NEG(N)/MZHG079(S) | 9 | 20.00 | 17.50 | 22.50 | 22.50 |
| NEG(N)/MZHG07C(S) | 9 | 15.00 | 15.00 | 20.00 | 17.50 |
| NEG(N)/MZHG07H(S) | 9 | 15.00 | 25.00 | 22.50 | 22.50 |
| NEG(N)/MZHG07J(S) | 9 | 50.00 | 60.00 | 60.00 | 62.50 |
| NEG(N)/MZHG07K(S) | 9 | 5.00 | 5.00 | 10.00 | 12.50 |
| NEG(N)/MZHG07M(S) | 9 | 0.00 | 5.00 | 2.50 | 2.50 |
| NEG(N)/MZHG07T(S) | 9 | 15.00 | 12.50 | 22.50 | 22.50 |
| NEG/MZHG045 | 10 | 30.00 | 30.00 | 30.00 | 30.00 |
| MZHG022 | 8 | 12.50 | 15.00 | 12.50 | 10.00 |
| MZHG02D | 8 | 40.00 | 35.00 | 35.00 | 40.00 |
| MZHG02P | 8 | 40.00 | 40.00 | 40.00 | 45.00 |
| MZHG02S | 8 | 10.00 | 20.00 | 15.00 | 15.00 |
| NEG/MZHG04F | 8 | 10.00 | 10.00 | 10.00 | 12.50 |
| NEG/MZHG07I | 9 | 20.00 | 20.00 | 20.00 | 27.50 |
| NEG/MZHG07V | 9 | 12.50 | 20.00 | 17.50 | 17.50 |

It is clear from these results that the synthetic plant promoters embodied in SEQ ID NO: 8 and SEQ ID NO: 9 function at least as well on average as the unmodified cestrum virus promoter. Additionally, SEQ ID NOs: 8 and 9 show no evidence of HDGS and HDMS. If there were silencing, these plants would not be as tolerant to glyphosate as the unmodified prCMP. Secondly, maize histone H3 and H4 genes are organized into multigene families of 40-50 and 50-60 copies, respectively. HDGS may be induced by the use of repetitive promoter or cis-elements. However, as maize already has 40-50 copies of endogenous histone promoter cis-elements, the potential to induce HDGS with either the wheat or maize H3 elements is unlikely (Chaubet et al., (1987) Histone genes in higher plants: organization and expression. Developmental Genetics 8: 461-473).

In view of the results presented here, an embodiment of the present invention is a synthetic plant promoter functional in a plant cell, wherein a 5′ terminus of the synthetic plant promoter is an enhancer from figwort mosaic virus or an enhancer from tobacco mosaic virus and wherein a 3′ terminus of the synthetic plant promoter is an enhancer from the tobacco mosaic virus when the 5′ terminus is the enhancer from figwort mosaic virus or the 3′ terminus is the enhancer from the figwort mosaic virus when the 5′ terminus is the enhancer from the tobacco mosaic virus. In another embodiment, the synthetic plant promoter has an optional Kozak sequence which extends beyond the 3′ terminus of the synthetic promoter. In another embodiment of the present invention, the enhancer from a figwort mosaic virus comprises SEQ ID NO: 1 and the enhancer from a tobacco mosaic virus comprises SEQ ID NO: 2. In yet another embodiment of the present invention, the synthetic plant promoter comprises any of SEQ ID NO: 3, 4, 5, 6, 7, 8, or 9.

An embodiment of the present invention is a method of constructing a synthetic plant promoter functional in a plant comprising the steps of: (a) obtaining an enhancer from a figwort mosaic virus and an enhancer from a tobacco mosaic virus and optionally one or more nucleotide sequences selected from the group consisting of enhancers, promoters, exons, introns, and other regulatory sequences; and (b) operably linking the enhancer from the figwort mosaic virus, the one or more optional nucleotide sequences, and the enhancer from the tobacco mosaic virus thus creating the synthetic plant promoter functional in a plant, wherein a 5′ terminus of the synthetic plant promoter is the enhancer from figwort mosaic virus or the enhancer from tobacco mosaic virus and wherein a 3′ terminus of the synthetic plant promoter is the enhancer from a tobacco mosaic virus when the said 5′ terminus is the enhancer from the figwort mosaic virus or the 3′ terminus of the promoter is the enhancer from the figwort mosaic virus when the said 5′ terminus is the enhancer from the tobacco mosaic virus, and wherein the one or more optional nucleotide sequences are positioned between the enhancers. Another embodiment of the present invention provides the method above, wherein the enhancer from the figwort mosaic virus comprises SEQ ID NO: 1 and the enhancer from the tobacco mosaic virus comprises SEQ ID NO: 2. In yet another embodiment, the product of step (b) comprises SEQ ID NO: 3. In still yet another embodiment, the product of step (b) comprises SEQ ID NO: 4. In another embodiment, the product of step (b) comprises SEQ ID NO: 5. In yet another embodiment, the product of step (b) comprises SEQ ID NO: 6. In still yet another embodiment, the product of step (b) comprises SEQ ID NO: 7. In further yet another embodiment, the product of step (b) comprises SEQ ID NO: 8. In another embodiment, the product of step (b) comprises SEQ ID NO: 9.

An embodiment of the present invention is a method of expressing a heterologous gene in a plant, plant cell, or plant tissue, comprising: (a) constructing a synthetic plant promoter according to the method of constructing a synthetic plant promoter functional in a plant comprising the steps of: (i) obtaining an enhancer from a figwort mosaic virus and an enhancer from a tobacco mosaic virus and optionally one or more nucleotide sequences selected from the group consisting of enhancers, promoters, exons, introns, and other regulatory sequences; and (ii) operably linking the enhancer from the figwort mosaic virus, the one or more optional nucleotide sequences, and the enhancer from the tobacco mosaic virus thus creating the synthetic plant promoter functional in a plant, wherein a 5′ terminus of the synthetic plant promoter is the enhancer from figwort mosaic virus or the enhancer from tobacco mosaic virus and wherein a 3′ terminus of the synthetic plant promoter is the enhancer from a tobacco mosaic virus when the said 5′ terminus is the enhancer from the figwort mosaic virus or the 3′ terminus of the promoter is the enhancer from the figwort mosaic virus when the said 5′ terminus is the enhancer from the tobacco mosaic virus, and wherein the one or more optional nucleotide sequences are positioned between the enhancers; (b) operably linking the synthetic plant promoter to the heterologous gene, thereby creating an expression cassette, wherein the expression cassette is functional in a plant, plant cell, or plant tissue; and (c) creating a plant, plant cell, or plant tissue or a portion thereof comprising the expression cassette, wherein the heterologous gene is expressed. In another embodiment, the heterologous gene comprises a nucleotide sequence encoding an herbicide resistance trait. In yet another embodiment, the nucleotide sequence encoding an herbicide resistance trait comprises a nucleotide sequence encoding HPPD resistance. In still yet another embodiment, the synthetic plant promoter is manipulated to optimize expression. In another embodiment, the synthetic plant promoter is manipulated to reduce expression. In further yet another embodiment, the synthetic plant promoter is manipulated to increase expression. In still yet another embodiment, the plant, plant cell, or plant tissue or a portion thereof is a monocot. In another embodiment, the plant, plant cell, or plant tissue or a portion thereof is maize. In yet another embodiment, the plant, plant cell, or plant tissue or a portion thereof is a dicot. In still yet another embodiment, the plant, plant cell, or plant tissue or a portion thereof is soybean.

An embodiment of the present invention is a method of selecting for male sterile plants comprising: (a) constructing an expression cassette comprising a synthetic plant promoter operably linked to a heterologous gene, wherein a 5′ terminus of the synthetic plant promoter comprises SEQ ID NO: 1 or SEQ ID NO: 2 and wherein a 3′ terminus of the synthetic plant promoter comprises SEQ ID NO: 2 when the 5′ terminus is SEQ ID NO: 1 or the 3′ terminus of the synthetic plant promoter is SEQ ID NO: 1 when the 5′ terminus is SEQ ID NO: 2, and wherein the synthetic plant promoter is functional in a plant cell; (b) creating a plant, plant cell, or plant tissue or a portion thereof comprising the expression cassette, wherein the heterologous gene is overexpressed and wherein such overexpression induces male sterility; and (c) selecting for the male sterile plants. In another embodiment, the synthetic plant promoter is selected from the group consisting of: SEQ ID NOs: 4 and 6. In yet another embodiment, the heterologous gene comprises a nucleotide sequence encoding an herbicide resistance trait. In still yet another embodiment, the nucleotide sequence encoding an herbicide resistance trait comprises a nucleotide sequence encoding HPPD resistance.

An embodiment of the present invention is a method of selecting for heterozygous plants comprising: (a) constructing an expression cassette comprising a synthetic plant promoter operably linked to a heterologous gene, wherein a 5′ terminus of the synthetic plant promoter comprises SEQ ID NO: 1 or SEQ ID NO: 2 and wherein a 3′ terminus of the synthetic plant promoter comprises SEQ ID NO: 2 when the 5′ terminus is SEQ ID NO: 1 or the 3′ terminus of the synthetic plant promoter is SEQ ID NO: 1 when the 5′ terminus is SEQ ID NO: 2, and wherein the synthetic plant promoter is functional in a plant cell; (b) creating a plant, plant cell, or plant tissue or a portion thereof comprising the expression cassette, wherein the heterologous gene is overexpressed in homozygous plants and wherein such overexpression induces gene silencing; and (c) selecting for the heterozygous plants. In another embodiment, the synthetic plant promoter is selected from the group consisting of: SEQ ID NOs: 4 and 6. In yet another embodiment, the heterologous gene comprises a nucleotide sequence encoding an herbicide resistance trait. In still yet another embodiment, the nucleotide sequence encoding an herbicide resistance trait comprises a nucleotide sequence encoding HPPD resistance.

### REFERENCES

Iyer M., Wu L., et al. V (2001) Two step transcriptional amplification as a method for imaging reporter gene expression using weak promoters PNAS 98(25):14595-14600.
Larkin, J. C., Oppenheimer, D. G., Pollock, S., and Marks, M. D. (1993) Arabidopsis GLABROUS1 gene requires downstream sequences for function. Plant Cell. 5(12): 1739-1748.
Sieburth, L. E., and Meyerowitz, E. M. (1997) Molecular dissection of the AGAMOUS control region shows that cis elements for spatial regulation are located intragenically. Plant Cell. 9(3): 355-365.
Batzer, et al (1991) Enhanced evolutionary PCR using oligonucleotides with inosine at the 3′-terminus. Nucleic Acid Res. 19:5081.
Ohtsuka, et al (1985) An alternative approach to deoxyoligonucleotides as hybridization probes by insertion of deoxyinosine at ambiguous codon positions. J. Biol. Chem. 260:2605-2608.
Rossolini, et al (1994) Use of deoxyinosine-containing primers vs degenerate primers for polymerase chain reaction based on ambiguous sequence information. Mol. Cell Probes 8:91-98.
Paszkowski et al (1984). Direct Gene Transfer to Plants. EMBO J 3:2717-2722
Potrykus et al (1985) Molecular and general genetics of a hybrid foreign gene introduced into tobacco by direct gene transfer. Mol. Gen. Genet. 199:169-177
Reich et al (1986) Efficient transformation of alfalfa protoplasts by the intranuclear microinjection of Ti-plasmids. Bio/Technology 4:1001-1004
Klein et al (1987) High velocity microprojectiles for delivering nucleic acids into living cells. Nature 327:70-73.
Uknes et al (1993) Regulation of pathogenesis-related protein-1a gene expression in tobacco. Plant Cell 5:159-169
Hofgen, R, and Willmitzer, L (1988) Storage of competent cells for Agrobacterium transformation. Nucl. Acid Res. 16:9877
Schocher et al (1986) Co-transformation of foreign genes into plants. Bio/Technology 4:1093-1096
Gordon-Kamm et al (1990) Transformation of Maize Cells and Regeneration of Fertile Transgenic Plants. Plant Cell 2:603-618
Fromm et al (1990) Inheritance and expression of chimeric genes in the progeny of transgenic maize plants. Bio/Technology 8:833-839.
Koziel et al (1993) Field performance of elite transgenic maize plants expressing an insecticidal protein derived from Bacillus thuringiensis. Bio/Technology 11:194-200
Vasil et al (1992) Herbicide resistant fertile transgenic wheat plants obtained by microprojectile bombardment of regenerable embryogenic callus. Bio/Technology 10:667-674
Vasil et al (1993) Rapid production of transgenic plants by direct bombardment of cultured immature embryos. Bio/Technology 11:1553-1558
Weeks et al (1993) Rapid Production of Multiple Independent Lines of Fertile Transgenic Wheat (Triticum aestivum). Plant Physiol. 1102:1077-1084
Murashiga et al (1962) A revised medium for rapid growth and bio-essays with tobacco tissue cultures. Physiologia Plantarum 15:473-497
Negrotto et al (2000) The use of phosphomannose isomerase as a selectable marker to recover transgenic maize plants (Zea mays L.) via Agrobacterium transformation. Plant Cell Reports 19:798-803
Eastmond, P. J., van Dijken, A. J. H., Spielman, M., Kerr, A., Tissier, A. F., Dickinson, H. G., Jones, J. D. G., Smeekens, S. C., Graham, I. A. (2002). Trehalose-6-phosphate synthase 1, which catalyses the first step in trehalose synthesis, is essential for Arabidopsis embryo maturation. Plant J. 29, 225-235.
Nuccio, M. L., Russell, B. L., Nolte, K. D., Rathinasabapathi, B., Gage, D. A., Hanson, D. A. (1998). The endogenous choline supply limits glycine betaine synthesis in transgenic tobacco expressing choline monooxygenase. Plant J. 16, 487-496.
Ranocha, P., McNeil, S. D., Ziemak, M. J., Li, C., Tarczynski, M. C., and Hanson, A. D. (2001). The S-methylmethionine cycle in angiosperms: ubiquity, antiquity and activity. Plant J. 25, 575-584.
Ritchie, S. W., Hanway, J. J., Benson, G. O. (1997). How a Corn Plant Develops. Special Report No. 48. Iowa State University of Science and Technology Cooperative Extension Service. Ames, Iowa.
Rontein, D., Dieuaide-Noubhani, M., Dufourc, E. J., Raymond, P., Rolin, D. (2002b). The metabolic architecture of plant cells. Stability of central metabolism and flexibility of anabolic pathways during the growth cycle of tomato cells. J. Biol. Chem. 277, 42948-43960.
Vogel, G., Aeschbacher, R. A., Müller, J., Boller, T. and Wiemken, A. (1998). Trehalose-6-phosphate phosphatases from Arabidopsis thaliana: identification by functional complementation of the yeast tps2 mutant. Plant J. 13, 673-683.
Wingler, A. (2002). The function of trehalose biosynthesis in plants. Phytochem. 60, 437-440.
Armenta, R. T. Tarnowski, I. Gibbons, and E. F. Ullman (1985) Improved Sensitivity in Homogeneous Enzyme Immunoassays using a Fluorogenic Macromolecular Substrate: an Assay for Serum Ferritin, Analytical Biochemistry, 146:211-219.
Ebert P., Ha S., An, G., Identification of an essential upstream element in the nopaline synthase promoter by stable and transient assays. Proc. Natl. Acad. Sci. USA 84:5745-5749 (1987)
Lawton M., Tierney M, Nakamura I., Anderson E., Komeda Y., Dube P., Hoffman N., Fraley R., Beachy R., Expression of a soybean β-conclycinin gene under the control of the Cauliflower Mosaic Virus 35S and 19S promoters in transformed petunia tissues. Plant Mol. Biol. 9:315-324 (1987)
Odell J., Nagy F., Chua N., Identification of DNA sequences required for activity of the cauliflower mosaic virus 35S promoter. Nature 313:810-812 (1985)
Sanger M., Daubert S., Goodman R., Characteristics of a strong promoter from figwort mosaic virus: comparison with the analogous 35S promoter from cauliflower mosaic virus and the regulated mannopine synthase promoter. Plant Mol. Biol. 14, 433-43 (1990)
Pellegrineschi A., Kis M., Dix I., Kavanagh T., Dix P., Expression of horseradish peroxidase in transgenic tobacco. Biochem. Soc. Trans. 23(2):247-250 (1995)
Walker J., Howard E., Dennis, E., Peacock W., DNA sequences required for anaerobic expression of the maize alcohol dehydrogenase 1 gene. Proc. Natl. Acad. Sci. USA 84:6624-6628 (1987)
Yang N., Russell D., Maize sucrose synthase-1 promoter directs phloem cell-specific expression of Gus gene in transgenic tobacco plants. Proc. Natl. Acad. Sci. USA 87:4144-8 (1990)
Chandler V., Radicella J., Robbins T., Chen J., Turks D., Two regulatory genes of the maize anthocyanin pathway are homologous: isolation of B utilizing R genomic sequences. Plant Cell 1:1175-1183 (1989)
Batzer M., Carlton J., Deininger P., Enhanced evolutionary PCR using oligonucleotides with inosine at the 3′-terminus. Nucleic Acid Res. 19:5081 (1991)
Ohtsuka E., Matsuki S., Ikehara M., Takahashi Y., Matsubara K., An alternative approach to deoxyoligonucleotides as hybridization probes by insertion of deoxyinosine at ambiguous codon positions. J. Biol. Chem. 260:2605-2608 (1985)
Rossolini G., Cresti S., Ingianni A., Cattani P., Riccio M., Satta G., Use of deoxyinosine-containing primers vs. degenerate primers for polymerase chain reaction based on ambiguous sequence information. Mol. Cell. Probes 8:91-98 (1994)
Ingelbrecht I., Herman L., Dekeyser R., Van Montagu M., Depicker A., Different 3′ end regions strongly influence the level of gene expression in plant cells. Plant Cell 1:671-680 (1989)
Sambrook, J. et al., In Molecular Cloning: A Laboratory Manual; 2^{nd} ed.; Cold Spring Harbor Laboratory Press Cold Spring Harbor, N.Y., 1989
Ausubel, F. M., Brent, R., Kingston, R. E., Moore, D. D., Seidman, J. G., Smith, J. A. and Struhl, K., Eds.; In Current Protocols in Molecular Biology; John Wiley and Sons: New York, 1990
Geiser M., Schweitzer S., Grimm C., The hypervariable region in the genes coding for entomopathogenic crystal proteins of Bacillus thuringiensis: nucleotide sequence of the kurhd1 gene of subsp. kurstaki HD1. (1986) Gene 48:109-118
Van Damme E., Smeets K., Van Leuven F., Peumans W., Molecular cloning of mannose-binding lectins from Clivia miniata. (1994) Plant Mol. Biol. 24:825-830
Castle L., Siehl D., Gorton R., Patten P., Chen Y., Bertain S., Cho H., Duck N., Wong J., Liu D., Lassner M. Discovery and directed evolution of a glyphosate tolerance gene. (2004) Science 304, 1151-1154
Cheng M., Jarret R., Zhijian L., Xing A., Demski J., Production of fertile transgenic peanut (Arachis hypogaea L.) plants using Agrobacterium tumefaciens. Plant Cell Rep. 15:653-657 (1996)
Cheng M., Fry J., Pang S., Zhou H., Hironaka C., Duncan D., Conner T., Wan Y., Genetic Transformation of Wheat Mediated by Agrobacterium tumefaciens. Plant Cell Rep. 15:971-980 (1997)
McKently A., Moore G., Doostdar H., Neidz R., Agrobacterium-mediated transformation of peanut (Arachis hypogaea L.) embryo axes and the development of transgenic plants. Plant Cell Rep. 14:699-703 (1995)
Newell, C. A., Plant Transformation Technology. Mol. Biotechnol. 16:53-65 (2000)
Weissbach and Weissbach, Eds.; In Methods for Plant Molecular Biology; Academic Press, Inc.: San Diego, Calif., 1988
Maliga et al., In Methods in Plant Molecular Biology; Cold Spring Harbor Press, 1995
Birren et al., In Genome Analysis: Detecting Genes, 1; Cold Spring Harbor: New York, 1998
Birren et al., In Genome Analysis: Analyzing DNA, 2; Cold Spring Harbor: New York, 1998
Clark, Ed., In Plant Molecular Biology: A Laboratory Manual; Springer: New York, 1997
Jones J., Dunsmuir P., Bedbrook J., High level expression of introduced chimaeric genes in regenerated transformed plants. EMBO J. 4:2411-2418 (1985)
De Almeida E., Gossele V., Muller C., Dockx J., Reynaerts A., Botterman J., Krebbers E., Timko M., Transgenic expression of two marker genes under the control of an Arabidopsis rbcS promoter: Sequences encoding the Rubisco transit peptide increase expression levels. Mol. Gen. Genetics 218:78-86 (1989)

What is claimed is:

1. A synthetic plant promoter functional in a plant cell, wherein a 5′ terminus of the synthetic plant promoter is an enhancer from figwort mosaic virus or an enhancer from tobacco mosaic virus and wherein a 3′ terminus of the synthetic plant promoter is an enhancer from the tobacco mosaic virus when the 5′ terminus is the enhancer from figwort mosaic virus or the 3′ terminus is the enhancer from the figwort mosaic virus when the 5′ terminus is the enhancer from the tobacco mosaic virus.

2. The synthetic plant promoter of claim 1, wherein an optional Kosak sequence extends beyond the 3′ terminus of the synthetic plant promoter.

3. The synthetic plant promoter of claim 1, wherein the enhancer from a figwort mosaic virus comprises SEQ ID NO: 1 and the enhancer from a tobacco mosaic virus comprises SEQ ID NO: 2.

4. The synthetic plant promoter of claim 3, wherein the synthetic plant promoter comprises SEQ ID NO: 3.

5. The synthetic plant promoter of claim 3, wherein the synthetic plant promoter comprises SEQ ID NO: 4.

6. The synthetic plant promoter of claim 3, wherein the synthetic plant promoter comprises SEQ ID NO: 5.

7. The synthetic plant promoter of claim 3, wherein the synthetic plant promoter comprises SEQ ID NO: 6.

8. The synthetic plant promoter of claim 3, wherein the synthetic plant promoter comprises SEQ ID NO: 7.

9. The synthetic plant promoter of claim 3, wherein the synthetic plant promoter comprises SEQ ID NO: 8.

10. The synthetic plant promoter of claim 3, wherein the synthetic plant promoter comprises SEQ ID NO: 9.

11. A method of constructing a synthetic plant promoter functional in a plant comprising the steps of:
a) obtaining an enhancer from a figwort mosaic virus and an enhancer from a tobacco mosaic virus and optionally one or more nucleotide sequences selected from the group consisting of enhancers, promoters, exons, introns, Kozak sequences, and other regulatory sequences;
b) operably linking the enhancer from the figwort mosaic virus, the one or more optional nucleotide sequences, and the enhancer from the tobacco mosaic virus thus creating the synthetic plant promoter functional in a plant, wherein a 5′ terminus of the synthetic plant promoter is the enhancer from figwort mosaic virus or the enhancer from tobacco mosaic virus and wherein a 3′ terminus of the synthetic plant promoter is the enhancer from a tobacco mosaic virus when the said 5′ terminus is the enhancer from the figwort mosaic virus or the 3′ terminus of the synthetic plant promoter is the enhancer from the figwort mosaic virus when the said 5′ terminus is the enhancer from the tobacco mosaic virus, and wherein the one or more optional nucleotide sequences are positioned between the enhancers.

12. The method of claim 11, wherein the enhancer from the figwort mosaic virus comprises SEQ ID NO: 1 and the enhancer from the tobacco mosaic virus comprises SEQ ID NO: 2.

13. The method of claim 11, wherein the product of step (b) comprises SEQ ID NO: 3.

14. The method of claim 11, wherein the product of step (b) comprises SEQ ID NO: 4.

15. The method of claim 11, wherein the product of step (b) comprises SEQ ID NO: 5.

16. The method of claim 11, wherein the product of step (b) comprises SEQ ID NO: 6.

17. The method of claim 11, wherein the product of step (b) comprises SEQ ID NO: 7.

18. The method of claim 11, wherein the product of step (b) comprises SEQ ID NO: 8.

19. The method of claim 11, wherein the product of step (b) comprises SEQ ID NO: 9.

20. A method of expressing a heterologous gene in a plant, plant cell, or plant tissue, comprising:
a) constructing a synthetic plant promoter according to the method of claim 10;
b) operably linking the synthetic plant promoter to the heterologous gene, thereby creating an expression cassette, wherein the expression cassette is functional in a plant, plant cell, or plant tissue; and
c) creating a plant, plant cell, or plant tissue or a portion thereof comprising the expression cassette, wherein the heterologous gene is expressed.

21. The method of claim 20, wherein the heterologous gene comprises a nucleotide sequence encoding an herbicide resistance trait.

22. The method of claim 21, wherein the nucleotide sequence encoding an herbicide resistance trait comprises a nucleotide sequence encoding HPPD resistance.

23. The method of claim 20, wherein the synthetic plant promoter is manipulated to optimize expression.

24. The method of claim 20, wherein the synthetic plant promoter is manipulated to reduce expression.

25. The method of claim 20, wherein the synthetic plant promoter is manipulated to increase expression.

26. The method of claim 20, wherein the plant, plant cell, or plant tissue or a portion thereof is a monocot.

27. The method of claim 26, wherein the plant, plant cell, or plant tissue or a portion thereof is maize.

28. The method of claim 20, wherein the plant, plant cell, or plant tissue or a portion thereof is a dicot.

29. The method of claim 28, wherein the plant, plant cell, or plant tissue or a portion thereof is soybean.

30. A method of selecting for male sterile plants comprising:
a) constructing an expression cassette comprising a synthetic plant promoter operably linked to a heterologous gene, wherein a 5′ terminus of the synthetic plant promoter comprises SEQ ID NO: 1 or SEQ ID NO: 2 and wherein a 3′ terminus of the synthetic plant promoter comprises SEQ ID NO: 2 when the 5′ terminus is SEQ ID NO: 1 or the 3′ terminus of the synthetic plant promoter is SEQ ID NO: 1 when the 5′ terminus is SEQ ID NO: 2, and wherein the synthetic plant promoter is functional in a plant cell;
b) creating a plant, plant cell, or plant tissue or a portion thereof comprising the expression cassette, wherein the heterologous gene is overexpressed and wherein such overexpression induces male sterility; and
c) selecting for the male sterile plants.

31. The method of claim 30, wherein the synthetic plant promoter is selected from the group consisting of: SEQ ID NOs: 4 and 6.

32. The method of claim 30, wherein the heterologous gene comprises a nucleotide sequence encoding an herbicide resistance trait.

33. The method of claim 32, wherein the nucleotide sequence encoding an herbicide resistance trait comprises a nucleotide sequence encoding HPPD resistance.

34. A method of selecting for heterozygous plants comprising:
a) constructing an expression cassette comprising a synthetic plant promoter operably linked to a heterologous gene, wherein a 5′ terminus of the synthetic plant promoter comprises SEQ ID NO: 1 or SEQ ID NO: 2 and wherein a 3′ terminus of the synthetic plant promoter comprises SEQ ID NO: 2 when the 5′ terminus is SEQ ID NO: 1 or the 3′ terminus of the synthetic plant promoter is SEQ ID NO: 1 when the 5′ terminus is SEQ ID NO: 2, and wherein the synthetic plant promoter is functional in a plant cell;
b) creating a plant, plant cell, or plant tissue or a portion thereof comprising the expression cassette, wherein the heterologous gene is overexpressed in homozygous plants and wherein such overexpression induces gene silencing; and
c) selecting for the heterozygous plants.

35. The method of claim 34, wherein the synthetic plant promoter is selected from the group consisting of: SEQ ID NOs: 4 and 6.

36. The method of claim 34, wherein the heterologous gene comprises a nucleotide sequence encoding an herbicide resistance trait.

37. The method of claim 36, wherein the nucleotide sequence encoding an herbicide resistance trait comprises a nucleotide sequence encoding HPPD resistance.


SEQUENCE LISTING (10 sequences)

SEQ ID NO: 1 - DNA, 194 bp, Figwort mosaic virus
agctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaa

SEQ ID NO: 2 - DNA, 68 bp, Tobacco mosaic virus
gtatttttacaacaattaccaacaacaacaaacaacaaacaacattacaattactatttacaattaca

SEQ ID NO: 3 - DNA, 625 bp, Artificial Sequence
  synthetic plant promoter
agctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttctgcaggtcgactctagaggatcctataaataggaagttcatttcatttggagaggaaacctcgagtatttttacaacaattaccaacaacaacaaacaacaaacaacattacaattactatttacaattaca

SEQ ID NO: 4 - DNA, 267 bp, Artificial Sequence
  synthetic plant promoter
agctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagtatttttacaacaattaccaacaacaacaaacaacaaacaacattacaattactatttacaattaca

SEQ ID NO: 5 - DNA, 1017 bp, Artificial Sequence
  synthetic plant promoter
agctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttctgcaggtcgactctagaggatcttataaataggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcaggtggatcctataaataggaagttcatttcatttggagaggaaacctcgagtatttttacaacaattaccaacaacaacaaacaacaaacaacattacaattactatttacaattaca

SEQ ID NO: 6 - DNA, 1716 bp, Artificial Sequence
  synthetic plant promoter
agctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttctgcaggtcgactctagaggatctcaacaatattgagggttttactccaagtaaaattttagttttatttttaattatcataaacgacataaatataatatggaaagatcacaaatactgattaaaaactaaaatcatcaaaacgaaaaggaaaaaagaaaaaattgggttcaactctcatgagttattaaacattttaggttttaggcttaaatctttaaaaaaaatcagaactgaaaaacgaaaaattctaattttattttggactctgattcatagcttatgtcgcttatgtagttatgctagggatgaatctgtatttcgttaccgtaatgagagttcgatactctcttacttgttacgattctggagcatgttacatttttttctttccgtcaacaacaactttaatatggtaaaacaaaatttatttttatttggctggtcctactcaagacaaatcttctgccgacatcacataatcatattaaaaaccataacttctgccactctgtttttttttttttttgtaaccattaactgattggattttgatccatctcatctgattttttagctcaacaatttacttgcacattttctatttggttttatttatacttagttacatatatgattatcgaactagtatctctttataattaagtatttttctatttttttttaatttagatttttgtgaattcatttacagtagaaaactgtaaaaccatatggtctaattatagaatgaaaacttcaacgaatccatacaacttattggctaaatataataaatctgcttgaagcatattgttattatttagttggatttgacgatctctgactttaatgtataccgacataccctatgatttagatgttgatttttcccattcttaatatatccatgttaagagattccaccataacatatctaattatttgcattgtaataaatattatcattaaaaaaaaatacaactggacagctggctcgtcccattgtttcttacgtccaccaattacatttgttaaagcaaacttattagaacgttcatgtgtgagaagttggtgtcgacatgtgtctaaggtctatgtcagaaatcggatcctataaataggaagttcatttcatttggagaggaaacctcgagtatttttacaacaattaccaacaacaacaaacaacaaacaacattacaattactatttacaattaca

SEQ ID NO: 7 - DNA, 2108 bp, Artificial Sequence
  synthetic plant promoter
agctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttctgcaggtcgactctagaggatctcaacaatattgagggttttactccaagtaaaattttagttttatttttaattatcataaacgacataaatataatatggaaagatcacaaatactgattaaaaactaaaatcatcaaaacgaaaaggaaaaaagaaaaaattgggttcaactctcatgagttattaaacattttaggttttaggcttaaatctttaaaaaaaatcagaactgaaaaacgaaaaattctaattttattttggactctgattcatagcttatgtcgcttatgtagttatgctagggatgaatctgtatttcgttaccgtaatgagagttcgatactctcttacttgttacgattctggagcatgttacatttttttctttccgtcaacaacaactttaatatggtaaaacaaaatttatttttatttggctggtcctactcaagacaaatcttctgccgacatcacataatcatattaaaaaccataacttctgccactctgtttttttttttttttgtaaccattaactgattggattttgatccatctcatctgattttttagctcaacaatttacttgcacattttctatttggttttatttatacttagttacatatatgattatcgaactagtatctctttataattaagtatttttctatttttttttaatttagatttttgtgaattcatttacagtagaaaactgtaaaaccatatggtctaattatagaatgaaaacttcaacgaatccatacaacttattggctaaatataataaatctgcttgaagcatattgttattatttagttggatttgacgatctctgactttaatgtataccgacataccctatgatttagatgttgatttttcccattcttaatatatccatgttaagagattccaccataacatatctaattatttgcattgtaataaatattatcattaaaaaaaaatacaactggacagctggctcgtcccattgtttcttacgtccaccaattacatttgttaaagcaaacttattagaacgttcatgtgtgagaagttggtgtcgacatgtgtctaaggtctatgtcagaaatcggatccacctgaaataaaacaatagaacaagtagaaaccaatcagcgaacatataccaaatcaaaagccgtaagagaaatcaaaacaacaccaaagagaaacggatctaaacataagaaacctaaaacagagagaatcgaacaaagaaaacacaaaaattgaatagatcgtccttgaaaatcctaatttcacaatcaagcaagaaattacacagatgtaaacactacgaatcgatatcttagtaatcaggacaaaatttagaagctggattgacgaaacgaacaatattgtcaaaagcaatttatacaaaagattcaataatccacataacaaaaattggagatcagatacgaatcaaaaacaaaaagaatcagaaaatatacctatttataagatcctataaataggaagttcatttcatttggagaggaaacctcgagtatttttacaacaattaccaacaacaacaaacaacaaacaacattacaattactatttacaattaca

SEQ ID NO: 8 - DNA, 844 bp, Artificial Sequence
  synthetic plant promoter
agctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttctgccggacccaagctttcggccacgtcaccaatccgcggcattcctacccggatcgccatctcgaccgtccactccatccgcatccaacggcaagtacatgcctctcgtcccctcccgaaaaaccaaaacccaaacccgcccgctctctcggtgctcctcctatttaactccgccccgtccccttcttcctcctcaccccaatctccccagcccccaaccaccagagcctcctcctcccctccggcgcatcgacgacaagggatccacctcgagtatttttacaacaattaccaacaacaacaaacaacaaacaacattacaattactatttacaattacacatgtaaacc

SEQ ID NO: 9 - DNA, 1304 bp, Artificial Sequence
  synthetic plant promoter
agctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttctgccggaccctcatgagcggagaattaagggagtcacgttatgacccccgccgatgacgcgggacaagccgttttacgtttggaactgacagaaccgcaacgaagctttggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttggcatatgctggtggcagagggtgtgcttccctgcttgggcgggcacgctgggatggcgaaaattttcggaagcgctgggatggcgaaaattttcggaagcgctgggcgctggcgcgggcggcggacaactatgggtcgttggattgggaattgggatggagggcgagggtgtcgctgctggcgggggtcgatccgcgtgttgagcgatctgcgaggattatgtccactcacccgtcagtctataaatacttagcccctccctcattgttaagggagcaaaatctcagagagatagtcctagagagagaaagagagcaagtagcctagaagtggatccacctcgagtatttttacaacaattaccaacaacaacaaacaacaaacaacattacaattactatttacaattacacatgtaaacc

SEQ ID NO: 10 - DNA, 1761 bp, wildtype cestrum virus
  misc_feature (1756)..(1761): Kozak
aaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataactccactgacgtaagggatgacgaacaatcccactatccttcacccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatctgcagcggaccccggaccctcatgagcggagaattaagggagtcacgttatgacccccgccgatgacgcgggacaagccgttttacgtttggaactgacagaaccgcaacgaagctttggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtggatccacctcgagtatttttacaacaattaccaacaacaacaaacaacaaacaacattacaattactatttacaattacacatgtaaacc